(19) United States
(12) Patent Application Publication
(10) Pub. No.: US 2013/0197069 A1
(43) Pub. Date: Aug. 1, 2013

(54) METHODS FOR TREATING STRESS INDUCED EMOTIONAL DISORDERS

(71) Applicant: MASSACHUSETTS INSTITUTE OF TECHNOLOGY, CAMBRIDGE, MA (US)

(72) Inventors: ANDRE FISCHER, GOETTINGEN (DE); LI-HUEI TSAI, CAMBRIDGE, MA (US)

(73) Assignee: MASSACHUSETTS INSTITUTE OF TECHNOLOGY, CAMBRIDGE, MA (US)

(21) Appl. No.: 13/851,481

(22) Filed: Mar. 27, 2013

Related U.S. Application Data
(62) Division of application No. 12/599,518, filed on Sep. 20, 2010, now abandoned.
(60) Provisional application No. 60/959,353, filed on Jul. 13, 2007.

(51) Int. Cl. A61K 31/505; A61K 31/52; A61K 31/7088; A61K 31/365

(52) U.S. Cl. CPC A61K 31/505; A61K 31/365; A61K 31/52; A61K 31/7088
USPC 514/44 R; 514/272; 514/473; 514/263.4

(57) ABSTRACT

The invention relates to methods and products for treating emotional disorders such as stress induced emotional disorders, as well as related assays and kits. Methods include administering to a subject an effective amount of an agent for targeting the Rac1, Cdk5, p35, PAK-1 pathway to treat the emotional disorder. The agent for targeting the Rac1, Cdk5, p35, PAK-1 pathway may be, for instance, a Rac-1 inhibitor, a Cdk5 inhibitor, a PAK-1 activator, or p35 mobilizing agent.

### RELATED APPLICATIONS

This application is a division of U.S. patent application Ser. No. 12/599,518, filed Sep. 20, 2010, which application is a national stage of PCT/US2008/008517, filed Jul. 11, 2008, which application claims the benefit under 35 USC §119 of U.S. provisional application Ser. No. 60/959,353, filed Jul. 13, 2007, all of which are incorporated herein by reference.

### FEDERALLY SPONSORED RESEARCH

This invention was made with Government support under National Institutes of Health NS051874. The Government has certain rights to this invention.

### FIELD OF THE INVENTION

The invention relates to methods and products for treating emotional disorders. In particular the methods are accomplished by targeting the Rac1, Cdk5, p35, PAK-1 pathway.

### BACKGROUND OF INVENTION

The pathogenesis of emotional disorders, post-traumatic stress disorders in particular, often involves associative learning that links anxiogenic stimuli to certain life experiences^{1 2 3 4}. These disorders severely affect the life of patients and are an increasing burden to our societies¹. Treatment of such disorders generally involves the promotion of extinction processes which are defined as the reduction of an aversively-motivated behavior². Therefore, understanding the molecular mechanisms underlying extinction can help to develop therapeutic strategies for emotional disorders.

A well-established paradigm to investigate extinction in rodents is pavlovian fear conditioning. In this paradigm a single exposure of rodents to a novel context followed by an electric foot-shock elicits the acquisition of conditioned fear. On the basis of associative learning the animals display an inborn aversive freezing behavior upon re-exposure to the conditioned context. This form of contextual fear conditioning is hippocampus-dependent and leads to a long-lasting fear memory⁵. During extinction animals are repeatedly re-exposed to the conditioned context without receiving the foot-shock again (extinction trial) which eventually results in the decline of the aversive freezing behavior⁶.

At present, the molecular mechanisms underlying extinction are not well understood. A number of studies showed that some, but not all components of the molecular machinery which is required for the initial encoding of fear memories also regulate extinction.^{7 8 9,10} Recent studies indicate that synaptic remodeling, for example mediated by actin re-arrangement, may be important for extinction.^{6 3} Actin dynamics are intimately involved in synaptic plasticity, synapse formation and neuronal morphology.^{6 11 12} Interestingly, cyclin-dependent kinase 5 (Cdk5), has been described as an important regulator of synaptic function and actin dynamics.^{13,14 15 16}

### SUMMARY OF INVENTION

In some aspects the invention relates to methods of treating emotional disorders such as stress induced emotional disorders. A method for treating an emotional disorder by administering to a subject an effective amount of an agent for targeting the Rac1, Cdk5, p35, PAK-1 pathway to treat the emotional disorder is provided. In some embodiments the agent for targeting the Rac1, Cdk5, p35, PAK-1 pathway is a Rac-1 inhibitor, a Cdk5 inhibitor, a PAK-1 activator, or p35 mobilizing agent. The methods in some embodiments involve the administration of a second therapeutic, such as a Rac1 inhibitor, a Cdk5 inhibitor and a PAK-1 activator. The agents may be administered, for instance, orally, intravenously, cutaneously, subcutaneously, nasally, imtramuscularly, intraperitoneally or intracerebroventricularly.

In one embodiment the Rac-1 inhibitor is a direct Rac-1 inhibitor. In other embodiments the Rac-1 inhibitor is an indirect Rac-1 inhibitor. The Rac-1 inhibitor may be a Rac-1 activity inhibitor or a Rac-1 expression inhibitor. In some embodiments the Rac-1 inhibitor is NSC23760.

In other embodiments the Cdk5 inhibitor is a direct Cdk5 inhibitor. According to other embodiments the Cdk5 inhibitor is an indirect Cdk5 inhibitor, such as, for instance, a Rac-1 inhibitor. The Cdk5 inhibitor may be a Cdk5 activity inhibitor or a Cdk5 expression inhibitor. In some embodiments the Cdk5 inhibitor is butyrolactone or roscovitine.

The PAK-1 activator in some embodiments is a direct PAK-1 activator and in other embodiments is an indirect PAK-1 activator, such as for instance, a Cdk5 inhibitor or a Rac-1 inhibitor. The PAK-1 activator may be a PAK-1 activity activator or a PAK-1 expression activator.

In other aspects the invention is directed to a pharmaceutical composition comprising an agent for targeting the Rac1, Cdk5, p35, PAK-1 pathway and a pharmaceutically acceptable carrier in a formulation for delivery to brain tissue.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a series of graphs demonstrating that inhibition of hippocampal Cdk5 activity facilitates extinction. a. Mice (10/group) were subjected to extinction paradigm and injected (i.h.) with the Cdk5 inhibitor butyrolactone I (50 ng) immediately after E1-E3. When compared to the vehicle group, butyrolactone I injected mice showed reduced freezing behavior on E2 and E3 indicating facilitated extinction. b. The experiment was performed as described under (a), but a lower concentration (25 ng) of butyrolactone I was injected i.h. after E1-E3. Extinction was similar in vehicle and butyrolactone I injected mice. c. Similar results were obtained when mice were administered 12.5 ng butyrolactone I. d. Consistent with the data shown under (A) a single injection (i.h.) of 50 ng butyrolactone immediately after E1 facilitates extinction as indicated by a significant reduction of freezing behavior on E2 when compared to a vehicle group. Importantly, the reduced freezing behavior persists throughout extinction trials in the absence of further butyrolacone I injection. e. Mice were exposed to our extinction paradigm and injected with butyrolactone I (50 ng) 1 h after E1. This procedure facilitated extinction as indicated by a significant reduction of freezing behavior on E2 when compared to a vehicle group. f. Injection (i.h.) of butyrolacone I 3 h after E1 had no significant effect on extinction when compared to the vehicle group. *P<0.05 vs. vehicle group. n=9-10 mice/group. Error bars indicate S.E.M.

FIG. 2 is a series of graphs and an immunoblot demonstrating that increased Cdk5 activity in CK-p25 Tg mice impairs extinction. a. Experimental design; CK-p25 Tg (n=9) and control mice (n=9) were trained by contextual fear conditioning and p25 expression was induced 24 h later for 1 week. Afterwards all mice were exposed to 6 extinction trials before p25 production in CK-p25 Tg was repressed by reintroducing doxycycline to the diet. After 1 week all mice were subjected to another 5 extinction trials on consecutive days. b. Immunoblot analysis of hippocampal lysates from CK-p25 Tg and control mice. Anti p35 antibody (C-19) was employed to detected endogenous p25 and p25-GFP in CK-p25 Tg mice 1 week after p25 induction and 1 week after p25 repression, respectively. c. Cdk5 kinase activity was analyzed by p35 (C-19) immunoprecipitation assays using histone H1 as an in vitro substrate. Cdk5 activity was significantly increased after 1 week induction and decreased to baseline levels after 1 week of p25 repression. d. CK-p25 Tg and control mice were subjected to the experiment purloined under (a). As long as p25 was expressed in CK-p25 Tg leading to increased Cdk5 activity, extinction of freezing behavior was abolished. When p25 production was repressed and Cdk5 activity reduced to baseline levels, extinction occurred in the same mice. ***P<0.0001. n=3-5 for biochemical analysis. Error bars indicate S.E.M.

FIG. 3 is a series of graphs and immunoblots demonstrating that membrane association of Cdk5/p35 during extinction is regulated by Rac-1. a. Membrane and cytosolic fractions were prepared form the hippocampus of mice 0.5 h after exposure to E1, E3 or from naïve animals. We observed a significant reduction of membrane associated p35 that was accompanied by increased cytosolic p35 levels after E3. Synaptophysin (SVP) served as a loading control for the membrane fraction, whereas GAPDH was employed to verify equal loading in the cytosolic fraction. b. Membrane lysates prepared from mice 0.5 h after E3 and from naïve mice were subjected to Rac-1 kinase assay. Rac-1 activity was significantly decreased after E3. c. Hippocampal cytosolic and membrane fractions were prepared on E3 0.5 h after mice were injected (icy) with either vehicle or Rac-1 inhibitor. Immunoblot analysis showed a significant reduction of membrane-associated Cdk5/p35 when compared to vehicle-injected mice. d. Consistently application of Rac-1 inhibitor facilitates extinction. Mice (10/group) were subjected to our extinction paradigm and injected (icv) with Rac-1 inhibitor (NSC23760; 10 μg/μl) immediately after E1-E3. When compared to the vehicle group, Rac-1 inhibitor injected mice showed reduced freezing indicating facilitated extinction. n=3-5 for biochemical analysis. ***P<0.0001 vs. naive/vehicle group; *P<0.05 vs. naive/vehicle group. Error bars indicate S.E.M.

FIG. 4 is a series of graphs and immunoblots demonstrating that inhibition of Rac-1 facilitates extinction. a. Membrane and cytosolic fractions were prepared form the hippocampus of mice 0.5 h after exposure to E1, E3 or from naïve animals and analyzed for the levels of the Cdk5 substrate PAK-1 by immunoblotting. Levels of PAK-1_{Thr212} were significantly decreased in the membrane fraction after E3. b. The same lysates as under (A) were used to analyze the levels of pPAK-1_{T423}, a non-Cdk5 phosphorylation site that indicates active PAK-1. pPAK-1_{T423} levels were significantly increased after E3 in the membrane fraction. In the cytosolic fraction pPAK-1_{T423} levels decreased after E1 when compared to naïve mice but increased back to baseline levels after E3. c. Hippocampal cytosolic and membrane fractions were prepared on E3 0.5 h after mice were injected (icv) with either vehicle or Rac-1 inhibitor. Immunoblot analysis showed a significant reduction of membrane associated p35/Cdk5, pPAK-1_{T212} and PAK-1 levels. d. The same lysates as described under (c) were used to immunoprecipitate PAK-1. An HA antibody served a control for unspecific binding of proteins to the proteinA agarose beads. The precipitates were immunoblotted with antibodies against PAK-1 and p35. Much less p35 co-immunoprecipitated with PAK-1 in lysates from mice that were administered with Rac-1 inhibitor. Error bars indicate S.E.M.

FIG. 5 is a series of graphs and immunoblots demonstrating that Cdk5 affects PAK-1 activity during extinction. a. Experimental design. Microcanulae were implanted into the hippocampus of mice that were subjected to contextual fear conditioning. The Cdk5 inhibitor butyrolactone (50 ng) was injected immediately after exposure to E1 and hippocampal cytosolic and membrane fractions were prepared 0.5 h later. b. Mice injected with butyrolactone I showed significantly reduced levels of PAK-1_{T212} in the cytosolic fraction. c. Similarly, PAK-1_{T212} levels were reduced in the membrane fraction. Conversely, levels of pPAK-1_{T423} were significantly increased. d. Notably, butyrolactone injected mice also displayed a significant re-distribution of PAK-1 from the membrane to the cytosol when compared to vehicle injected mice. Error bars indicate S.E.M.

FIG. 6 is a graph and pictures demonstrating that PAK-1 activity promotes extinction. a. Experimental design. Mice were implanted with microcannulae into the dorsal hippocampus and subjected to contextual fear conditioning. After 24 h mice were injected with either HSV-GFP or HSV-GFP-dominant negative PAK-1 (dnPAK-1) virus into the dorsal hippocampus. As of the next day all mice were subjected to our extinction paradigm. b. Herpes Virus expressing GFP and a dominant negative PAK-1 (dnPAK-1) construct or only GFP were injected into the dorsal hippocampus of mice. Upper panel: DAB staining for GFP of mice injected with dnPAK-1/GFP. Lower panel: high magnification pictures of the hippocampal region showing staining for Hoechst, NeuN and GFP. The transfection efficiency was about 30%. Analysis was performed 48 h after injection. c. Mice (n=10/group) expressing dnPAK-1 in the hippocampus display a significantly impaired reduction of freezing behavior indicating impaired extinction. Error bars indicate S.E.M.

### DETAILED DESCRIPTION

Treatment of emotional disorders, as provided herein, involves the promotion of extinction processes, which are defined as the learned reduction of fear. The molecular mechanisms underlying extinction have only begun to be elucidated. By employing genetic and pharmacological approaches in mice, it is shown herein that extinction requires down-regulation of Rac-1 and cyclin-dependent kinase 5 (Cdk5) and up-regulation of p21 activated kinase-1 (PAK-1) activity. This is physiologically achieved by a Rac-1 dependent relocation of the Cdk5 activator p35 from the membrane to the cytosol and dissociation of p35 from PAK-1. Moreover, the data described herein imply that Cdk5/p35 activity prevents extinction in part by inhibition of PAK-1 activity in a Rac-1 dependent manner. Thus, extinction of contextual fear can be regulated by counteracting components of a molecular pathway involving Rac-1, Cdk5 and PAK-1. Targeting this pathway provides a suitable therapeutic avenue to treat emotional disorders.

The methods of the invention are accomplished using agents for targeting the Rac1, Cdk5, p35, PAK-1 pathway. An agent for targeting the Rac1, Cdk5, p35, PAK-1 pathway as used herein is any compound that modifies the activity or functional expression of Rac1, Cdk5, p35 or PAK-1 consistent with promoting extinction. Decreased levels of active Rac1, Cdk5 and membrane bound p35 are consistent with promoting extinction. Increased levels of active PAK-1 and cytosolic p35 are consistent with promoting extinction.

Rac1 is a member of the Rho family of low molecular weight GTPases and are related to each other based on sequence homology and function (Vojtek, A. B., and Cooper, J. A., Cell 1995, 82, 527-529). In an active state, they bind to GTP and transduce signals of other proteins in signal transduction pathways. In their inactive state, they are bound to GDP. Members of the Rho family are typically involved in regulation of the actin cytoskeleton.

Cdk5 is a serine/threonine protein-kinase critical for the development of the central nervous system¹⁷. Two related neuron-specific proteins, p35 and p39, are required and sufficient to activate Cdk5 upon direct binding¹⁴. Recently, there has been emerging evidence for a role of Cdk5/p35 in the adult brain. Cdk5 phosphorylates a number of synaptic proteins, and regulates dendritic spine morphogenesis, synaptic plasticity and learning^{18 19 20 21 22 23 24 25 26}. Cdk5 is believed to regulate actin dynamics via its upstream regulator Rac-1 and downstream target PAK-1, which have both also been implicated in synaptic plasticity^{27 28 15}. It has been demonstrated according to the invention that inhibition of Cdk5 in the hippocampus facilitates extinction of learned contextual fear. Conversely, extinction was severely impaired when Cdk5 activity was upregulated. During physiological extinction, Cdk5 activity was down-regulated by a reduced membrane-association of p35 via the small GTPase Rac-1. Furthermore, our data show that inhibition of PAK-1 activity by Cdk5 impedes extinction.

The inhibitors of the invention embrace compounds which are Rac1 or Cdk5 antagonists. Such inhibitors are referred to as activity inhibitors or Rac1 activity inhibitors or Cdk5 activity inhibitors. As used herein, an activity inhibitor is an agent which interferes with or prevents the activity of Rac1 or Cdk5. An activity inhibitor may interfere with the ability of the Rac1 or Cdk5 to interact with a native binder or ligand, such as p35. An activity inhibitor may be an agent which competes with a naturally occurring activator of Rac1 or Cdk5 for interaction with the activation site on the Rac1 or Cdk5. Alternatively, the activity inhibitor may bind to the Rac1 or Cdk5 at a site distinct from the activation binding site, but in doing so, it may, for example, cause a conformational change in the Rac1 or Cdk5 which is transduced to the activation binding site, thereby precluding binding of the natural activator. Alternatively, an activity inhibitor may interfere with a component upstream or downstream of the Rac1 or Cdk5 but which interferes with the activity of the Rac1 or Cdk5. This latter type of activity inhibitor is referred to as a functional antagonist.

In investigating the role of Rac-1 during Cdk5-dependent extinction it was discovered that extinction led to a reduction of membrane associated GTP bound Rac-1 after E3 without affecting the total amount of Rac-1 protein, implying reduced Rac-1 activity. Interestingly, when Rac-1 activity was inhibited during extinction a redistribution of both, p35 and Cdk5, from the membrane to the cytosol was observed while during physiological extinction only p35 was affected.

Examples of Rac1 inhibitors which are activity inhibitors include Rac1 Inhibitor W56, sold by Tocris Biosciences (Ellisville, Mo.), NSC23760 and NSC 23766 sold by EMD Biosciences (San Diego, Calif.) or the inhibitors described in Yuan Gao, et al. PNAS, May 18, 2004, vol. 101, 7618-7623. Rac1 Inhibitor W56 is a peptide comprising residues 45-60 of the guanine nucleotide exchange factor (GEF) recognition/activation site of Rac1; selectively inhibits Rac1 interaction with Rac1-specific GEFs TrioN, GEF-H1 and Tiam1 and is described in Gao et al (2001) J. Biol. Chem. 276 47530.

W56 has the following sequence Met-Val-Asp-Gly-Lys-Pro-Val-Asn-Leu-Gly-Leu-Trp-Asp-Thr-Ala-Gly (SEQ ID NO.1). One of skill in the art will recognize that conservative amino acid substitutions may be made without affecting structure and function of W56. As used herein, a “conservative amino acid substitution” or “conservative substitution” refers to an amino acid substitution in which the substituted amino acid residue is of similar charge as the replaced residue and is of similar or smaller size than the replaced residue. Conservative substitutions of amino acids include substitutions made amongst amino acids within the following groups: (a) the small non-polar amino acids, A, M, I, L, and V; (b) the small polar amino acids, G, S, T and C; (c) the amido amino acids, Q and N; (d) the aromatic amino acids, F, Y and W; (e) the basic amino acids, K, R and H; and (f) the acidic amino acids, E and D. Substitutions which are charge neutral and which replace a residue with a smaller residue may also be considered “conservative substitutions” even if the residues are in different groups (e.g., replacement of phenylalanine with the smaller isoleucine). The term “conservative amino acid substitution” also refers to the use of amino acid analogs or variants.

Examples of Cdk5 inhibitors which are activity inhibitors include butyrolactone I and roscovitine, for example. In the examples provided herein it is shown that pharmacological inhibition of hippocampal Cdk5 activity by butyrolactone I or roscovitine facilitates extinction in the contextual fear conditioning paradigm. Conversely, increased Cdk5 activity in CK-p25 Tg mice severely impaired extinction as indicated by the persistence of aversive freezing behavior. While Cdk5 activity seems to be important for the initial encoding of the contextual fear memory trace¹⁸, under the conditions of the CK-p25 Tg mice experiment Cdk5 appeared to subsequently impair its extinction. Notably, similar observations have been made for cAMP signaling. To this end cAMP is required for the acquisition of fear memory³⁸ but mice over-expressing the type I adenylyl cyclase Adcy1 in the forebrain, and display elevated hippocampal cAMP levels, show slower extinction of fear³⁹.

In order to elucidate the molecular mechanism by which Cdk5 activity regulates extinction we took advantage of the employed experimental extinction paradigm. As such after a single fear conditioning training mice display freezing behavior on E1-E3. However, when retested on E4 freezing was usually significantly decreased. We therefore speculated that the key molecular processes triggered after E1 (mice show freezing on subsequent E2) and E3 (mice show reduced freezing on subsequent E4) are different.

Other agents which are useful according to the methods of the invention in the treatment of conditions described herein include agents which interfere with Rac1 and Cdk5 expression at either the mRNA or protein level. Such inhibitors are referred to as expression inhibitors or Rac1 and Cdk5 expression inhibitors.

The inhibitors described herein are isolated molecules. An isolated molecule is a molecule that is substantially pure and is free of other substances with which it is ordinarily found in nature or in vivo systems to an extent practical and appropriate for its intended use. In particular, the molecular species are sufficiently pure and are sufficiently free from other biological constituents of host cells so as to be useful in, for example, producing pharmaceutical preparations or sequencing if the molecular species is a nucleic acid, peptide, or polysaccharide. Because an isolated molecular species of the invention may be admixed with a pharmaceutically-acceptable carrier in a pharmaceutical preparation, the molecular species may comprise only a small percentage by weight of the preparation. The molecular species is nonetheless substantially pure in that it has been substantially separated from the substances with which it may be associated in living systems.

Thus, in addition to the traditional inhibitors described above, agents for targeting the Rac1, Cdk5, p35, PAK-1 pathway can also be inhibited by antisense and RNAi mechanisms. Thus, the invention embraces antisense oligonucleotides that selectively bind to nucleic acid molecules encoding Rac1 or Cdk5 to decrease expression and activity of this protein.

As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Antisense oligonucleotides that selectively bind to a nucleic acid molecule encoding Rac1 or Cdk5 are particularly preferred. Those skilled in the art will recognize that the exact length of the antisense oligonucleotide and its degree of complementarity with its target will depend upon the specific target selected, including the sequence of the target and the particular bases which comprise that sequence.

It is preferred that the antisense oligonucleotide be constructed and arranged so as to bind selectively with the target under physiological conditions, i.e., to hybridize substantially more to the target sequence than to any other sequence in the target cell under physiological conditions. Based upon the nucleotide sequences of nucleic acid molecules encoding Rac1 or Cdk5, (e.g., GenBank Accession Nos AL590642, AL353736, AL391870, AL513188, AL513549, AL513015, AL512405, AL451080, AL121832, AL033521, NG_{—}005525, AL138836, AL355392, NM_{—}198829, NM_{—}006908, NM_{—}018890) or upon allelic or homologous genomic and/or cDNA sequences, one of skill in the art can easily choose and synthesize any of a number of appropriate antisense molecules for use in accordance with the present invention. In order to be sufficiently selective and potent for inhibition, such antisense oligonucleotides should comprise at least about 10 and, more preferably, at least about 15 consecutive bases which are complementary to the target, although in certain cases modified oligonucleotides as short as 7 bases in length have been used successfully as antisense oligonucleotides. See Wagner et al., Nat. Med. 1(11):1116-1118, 1995. Most preferably, the antisense oligonucleotides comprise a complementary sequence of 20-30 bases. Although oligonucleotides may be chosen which are antisense to any region of the gene or mRNA transcripts, in preferred embodiments the antisense oligonucleotides correspond to N-terminal or 5′ upstream sites such as translation initiation, transcription initiation or promoter sites. In addition, 3′-untranslated regions may be targeted by antisense oligonucleotides. Targeting to mRNA splicing sites has also been used in the art but may be less preferred if alternative mRNA splicing occurs. In addition, the antisense is targeted, preferably, to sites in which mRNA secondary structure is not expected (see, e.g., Sainio et al., Cell Mol. Neurobiol. 14(5):439-457, 1994) and at which proteins are not expected to bind.

In one set of embodiments, the antisense oligonucleotides of the invention may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art recognized methods which may be carried out manually or by an automated synthesizer. They also may be produced recombinantly by vectors.

In preferred embodiments, however, the antisense oligonucleotides of the invention also may include “modified” oligonucleotides. That is, the oligonucleotides may be modified in a number of ways which do not prevent them from hybridizing to their target but which enhance their stability or targeting or which otherwise enhance their therapeutic effectiveness.

The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acid molecules has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars which are covalently attached to low molecular weight organic groups other than a hydroxyl group at the 3′ position and other than a phosphate group at the 5′ position. Thus modified oligonucleotides may include a 2′-O-alkylated ribose group. In addition, modified oligonucleotides may include sugars such as arabinose instead of ribose.

The present invention, thus, contemplates pharmaceutical preparations containing modified antisense molecules that are complementary to and hybridizable with, under physiological conditions, nucleic acid molecules encoding Rac1 or Cdk5, together with pharmaceutically acceptable carriers. Antisense oligonucleotides may be administered as part of a pharmaceutical composition. In this latter embodiment, it may be preferable that a slow intravenous administration be used. Such a pharmaceutical composition may include the antisense oligonucleotides in combination with any standard physiologically and/or pharmaceutically acceptable carriers which are known in the art. The compositions should be sterile and contain a therapeutically effective amount of the antisense oligonucleotides in a unit of weight or volume suitable for administration to a subject.

The methods of the invention also encompass use of isolated short RNA that directs the sequence-specific degradation of an mRNA for Rac1 or Cdk5 through a process known as RNA interference (RNAi). The process is known to occur in a wide variety of organisms, including embryos of mammals and other vertebrates. It has been demonstrated that dsRNA is processed to RNA segments 21-23 nucleotides (nt) in length, and furthermore, that they mediate RNA interference in the absence of longer dsRNA. Thus, these 21-23 nt fragments are sequence-specific mediators of RNA degradation and are referred to herein as siRNA or RNAi. Methods of the invention encompass the use of these fragments (or recombinantly produced or chemically synthesized oligonucleotides of the same or similar nature) to enable the targeting of mRNAs for Rac1 or Cdk5 for degradation in mammalian cells useful in the therapeutic applications discussed herein.

The methods for design of the RNA's that mediate RNAi and the methods for transfection of the RNAs into cells and animals is well known in the art and are readily commercially available (Verma N. K. et al, J. Clin. Pharm. Ther., 28(5):395-404 (2004), Mello C. C. et al. Nature, 431(7006)338-42 (2004), Dykxhoorn D. M. et al., Nat. Rev. Mol. Cell. Biol. 4(6):457-67 (2003) Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK)). The RNAs are preferably chemically synthesized using appropriately protected ribonucleoside phosphoramidites and a conventional DNA/RNA synthesizer. Most conveniently, siRNAs are obtained from commercial RNA oligo synthesis suppliers listed herein. In general, RNAs are not too difficult to synthesize and are readily provided in a quality suitable for RNAi. A typical 0.2 μmol-scale RNA synthesis provides about 1 milligram of RNA, which is sufficient for 1000 transfection experiments using a 24-well tissue culture plate format.

The cDNA specific Rac1 or Cdk5 siRNA is designed preferably by selecting a sequence that is not within 50-100 bp of the start codon and the termination codon, avoids intron regions, avoids stretches of 4 or more bases such as AAAA, CCCC, avoids regions with GC content <30% or >60%, avoids repeats and low complex sequence, and it avoids single nucleotide polymorphism sites. The agent for targeting the Rac1, Cdk5, p35, PAK-1 pathway siRNA may be designed by a search for a 23-nt sequence motif AA(N19). If no suitable sequence is found, then a 23-nt sequence motif NA(N21) may be used with conversion of the 3′ end of the sense siRNA to TT. Alternatively, the Rac1 or Cdk5 siRNA can be designed by a search for NAR(N17)YNN. The target sequence may have a GC content of around 50%. The siRNA targeted sequence may be further evaluated using a BLAST homology search to avoid off target effects on other genes or sequences. Negative controls are designed by scrambling targeted siRNA sequences. The control RNA preferably has the same length and nucleotide composition as the siRNA but has at least 4-5 bases mismatched to the siRNA. The RNA molecules of the present invention can comprise a 3′ hydroxyl group. The RNA molecules can be single-stranded or double stranded; such molecules can be blunt ended or comprise overhanging ends (e.g., 5′, 3′) from about 1 to about 6 nucleotides in length (e.g., pyrimidine nucleotides, purine nucleotides). In order to further enhance the stability of the RNA of the present invention, the 3′ overhangs can be stabilized against degradation. The RNA can be stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′ hydroxyl significantly enhances the nuclease resistance of the overhang in tissue culture medium.

The RNA molecules used in the methods of the present invention can be obtained using a number of techniques known to those of skill in the art. For example, the RNA can be chemically synthesized or recombinantly produced using methods known in the art. Such methods are described in U.S. Published Patent Application Nos. US2002-0086356A1 and US2003-0206884A1 that are hereby incorporated by reference in their entirety.

The methods described herein are used to identify or obtain RNA molecules that are useful as sequence-specific mediators of Rac1 or Cdk5 mRNA degradation and, thus, for inhibiting Rac1 or Cdk5 activity. Expression of Rac1 or Cdk5 can be inhibited in humans in order to prevent the protein from being translated and thus contributing to the inhibition of extinction.

The RNA molecules may also be isolated using a number of techniques known to those of skill in the art. For example, gel electrophoresis can be used to separate RNAs from the combination, gel slices comprising the RNA sequences removed and RNAs eluted from the gel slices. Alternatively, non-denaturing methods, such as non-denaturing column chromatography, can be used to isolate the RNA produced. In addition, chromatography (e.g., size exclusion chromatography), glycerol gradient centrifugation, affinity purification with antibody can be used to isolate RNAs.

Any RNA can be used in the methods of the present invention, provided that it has sufficient homology to the Rac1 or Cdk5 genes to mediate RNAi. The RNA for use in the present invention can correspond to the entire Rac1 or Cdk5 genes or a portion thereof. There is no upper limit on the length of the RNA that can be used. For example, the RNA can range from about 21 base pairs (bp) of the gene to the full length of the gene or more. In one embodiment, the RNA used in the methods of the present invention is about 1000 bp in length. In another embodiment, the RNA is about 500 bp in length. In yet another embodiment, the RNA is about 22 bp in length. In certain embodiments the preferred length of the RNA of the invention is 21 to 23 nucleotides.

The agents of the invention also include activators of PAK-1. PAK-1 Serine/threonine p21-activated kinases (PAKs) mediate the signals of the Cdk5 and Rac1. The PAK family comprises at least four isoforms, PAK-1, PAK-2, PAK-3 and PAK-4, which are critical reorganization but are also involved in nuclear signaling. The PAK proteins have been implicated in a wide range of biological activities such as neurite formation and axonal guidance, development of cell polarity and motile responses. PAK-1, specifically activates the JNK1 MAP Kinase signaling pathway in mammals and can also function in place of Ste20, the analogous yeast protein which activates the yeast pheromone response MAP Kinase cascade and regulates cellular morphogenesis. The gene encoding PAK-1 is also referred to as p21-activated kinase 1, PAK-alpha, alpha-PAK, hPAK1, yeast Ste20-related, and CDC42/RAC1 effector.

PAK-1 contains a regulatory domain and a kinase catalytic domain which can interact intramolecularly resulting in a closed, inactive configuration. This autoinhibition is decreased in PAK1 containing mutations in the regulatory region. Mutants of PAK-1 have been generated in several places including amino terminal mutants, kinase dead mutants. A non-phosphorylatable mutant of PAK-1 lacking threonine 212 was expressed in neurons to demonstrate dramatic neurite disorganization.

PAK-1 activators include but are not limited to PAK-1 activity activators and PAK-1 expression activators. PAK-1 activity activators include, for instance, molecules which promote the phosphorylation of threonine 423 of PAK-1 or reduce the phosphorylation of Threonine 212. Cdk5 directly phosphorylates PAK-1 on threonine 212 and inhibits PAK1 activity. The data described in the examples demonstrating that PAK-1_{T212} levels decrease during extinction implied that PAK-1 activity might be up-regulated. In line with this assumption it was found according to the invention that phosphorylation of membrane associated PAK-1 at the non-Cdk5 site threonine 423 was increased after E3. The autophosphorylation of threonine 423 of PAK-1 liberates the Pak-1 inhibitory domain and is a marker for active PAK-1^{28,37} Cytosolic PAK-1_{T423} levels decreased after E1 but increased back to baseline after E3. These findings imply that the reduction of aversive freezing behavior correlates with increased PAK-1 activity.

Notably, it was found that inhibition of Rac-1 and Cdk5 activity during extinction reduced PAK-1_{T212} levels, providing direct evidence that Rac-1 and Cdk5 regulate PAK-1 through threonine 212 phosphorylation during extinction. Interestingly, inhibition of Cdk5 also led to increased PAK-1_{T423} levels. This data shows that Cdk5 activity regulates PAK-1 during extinction.

Other PAK-1 activity activators include agents that promote p35/Cdk5 sequestration. Inhibition of Rac-1 and Cdk5 induced a redistribution of PAK-1 from the membrane to the cytosol. This is consistent with previous findings demonstrating that Cdk5 activity affects the subcellular localization of PAK-1 in neuronal growth cones³⁶. However, despite increased cytosolic p35/Cdk5 levels there was no corresponding increase in the phosphorylation of PAK-1 on threonine 212 in the cytosol. It was actually reduced. One possible mechanism is that Cdk5/p35 is sequestered from PAK-1 upon re-distribution to the cytosol, because independent from phosphorylation, p35-PAK-1 interaction has been implicated with inhibition PAK-1 activity³⁶. Indeed it was discovered that less p35 co-immunoprecipitated with PAK-1 in hippocampal lysate prepared from mice injected with the Rac-1 inhibitor compared to the vehicle group.

Thus, during extinction membrane depletion of Cdk5 activity and dissociation of p35 from PAK-1 in the cytosol removes the inhibitory tone on PAK-1 activity. This eventually results in an increase of mainly cytosolic PAK-1 activity. It is likely that PAK-1 activity during extinction is regulated by additional mechanisms such as protein phosphatases. However, PAK-1 activators, as used herein, do not include protein phosphatases such as PP1⁴³ unless they are specifically recited in the claim. Direct evidence that PAK-1 activity is required for extinction is provided herein, because inhibition of PAK-1 by viral-mediated hippocampal expression of a dominant negative PAK-1 mutant significantly impaired extinction. As such, the effect of PAK-1 inhibition on extinction is opposite to that of Rac-1 and Cdk5 inhibition.

A PAK-1 expression activator as used herein is a compound capable of inducing the expression of an endogenous or exogenous PAK-1 or is a functional exogenous PAK-1. For instance, an expression vector carrying PAK-1 may be administered to the subject to induce the expression of PAK-1.

The PAK-1 nucleic acid may be operably linked to a gene expression sequence which directs the expression of the PAK-1 nucleic acid within a eukaryotic cell. The “gene expression sequence” is any regulatory nucleotide sequence, such as a promoter sequence or promoter-enhancer combination, which facilitates the efficient transcription and translation of the PAK-1 nucleic acid to which it is operably linked. The gene expression sequence may, for example, be a mammalian or viral promoter, such as a constitutive or inducible promoter. Constitutive mammalian promoters include, but are not limited to, the promoters for the following genes: hypoxanthine phosphoribosyl transferase (HPTR), adenosine deaminase, pyruvate kinase, and β-actin. Exemplary viral promoters which function constitutively in eukaryotic cells include, for example, promoters from the simian virus, papilloma virus, adenovirus, human immunodeficiency virus (HIV), Rous sarcoma virus, cytomegalovirus, the long terminal repeats (LTR) of moloney leukemia virus and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

In general, the gene expression sequence shall include, as necessary, 5′ non-transcribing and 5′ non-translating sequences involved with the initiation of transcription and translation, respectively, such as a TATA box, capping sequence, CAAT sequence, and the like. Especially, such 5′ non-transcribing sequences will include a promoter region which includes a promoter sequence for transcriptional control of the operably joined PAK-1 nucleic acid. The gene expression sequences optionally include enhancer sequences or upstream activator sequences as desired.

Preferably, the PAK-1 nucleic acid of the invention is linked to a gene expression sequence which permits expression of the PAK-1 nucleic acid in the cell. A sequence which permits expression of the PAK-1 nucleic acid in the cell is one which is selectively active in the particular cell and thereby causes the expression of the PAK-1 nucleic acid in the cells. Those of ordinary skill in the art will be able to easily identify promoters that are capable of expressing a PAK-1 nucleic acid in a cell based on the type of cell.

The PAK-1 nucleic acid sequence and the gene expression sequence are said to be “operably linked” when they are covalently linked in such a way as to place the transcription and/or translation of the PAK-1 coding sequence under the influence or control of the gene expression sequence. If it is desired that the PAK-1 sequence be translated into a functional protein, two DNA sequences are said to be operably linked if induction of a promoter in the 5′ gene expression sequence results in the transcription of the PAK-1 sequence and if the nature of the linkage between the two DNA sequences does not (1) result in the introduction of a frame-shift mutation, (2) interfere with the ability of the promoter region to direct the transcription of the PAK-1 sequence, or (3) interfere with the ability of the corresponding RNA transcript to be translated into a protein. Thus, a gene expression sequence would be operably linked to a PAK-1 nucleic acid sequence if the gene expression sequence were capable of effecting transcription of that PAK-1 nucleic acid sequence such that the resulting transcript might be translated into the desired protein or polypeptide.

The agents of the invention also include compounds that promote migration of p35 from the membrane to the cytosol. As described above, inhibition of Rac1 leads to p35 mobilization to the cytosol. Depletion of p35 from the membrane correlated with reduced phosphorylation of the Cdk5 substrate PAK-1 indicating that extinction leads to reduced membrane associated Cdk5 activity. Thus, membrane depletion of p35 is a mechanism to locally reduce Cdk5 activity, thereby allowing extinction to occur.

In some instances the Rac1 inhibitors, Cdk5 inhibitors and PAK-1 activators are direct inhibitors or activators and in other cases they are indirect inhibitors or activators. A direct activator or inhibitor is a molecule which interacts directly with the target. An indirect activator or inhibitor is one that interacts directly with an upstream effectors to produce an effect on the target.

RAC-1/Cdk5 and PAK-1 are counteracting components of a hippocampal signaling pathway that regulate extinction of contextual fear. The data presented below delineate a molecular pathway whereby extinction requires down-regulation of Rac/Cdk5 and up-regulation of PAK-1 activity. Modulators of these effectors are useful in the treatment of emotional disorders. Emotion disorders as used herein refer to disorder involving a fear based reaction to stimuli. An essential feature is a persistent fear of a circumscribed stimulus, Emotional disorders include but are not limited to post traumatic stress and phobias. Examples of definitions of these conditions and disorders can be found, for example, in the American Psychiatric Association: Diagnostic and Statistical Manual of Mental Disorders, Fourth Edition, Text Revision, Washington, D.C., American Psychiatric Association, 2000.

Post-Traumatic Stress Disorder is an anxiety neurosis caused by exposure to psychological damage by experience beyond a usual corrective ability such as traumas of wars, natural disasters, domestic violence or sexual abuse, etc. It is believed that in addition to psychological manifestations, shrinkage of the hippocampus and dysfunction of prefrontal cortex often occurs. In some instances the number of benzodiazepine receptors decreases. The principal characteristic symptoms involve re-experiencing a traumatic (i.e., psychologically distressing) event, the avoidance of stimuli associated with that event, the numbing of general responsiveness, and increased arousal. The “events” concerned are outside the range of common experiences such as simple bereavement, chronic illness and marital conflict.

Phobias include specific phobias and social phobias. Specific phobia is an anxiety disorder of which the essential feature is a persistent fear of a circumscribed stimulus, which may be an object or situation, other than fear of having a panic attack or of humiliation or embarrassment in social situations (which falls under social phobia). Examples include phobias of flying, heights, animals, injections, and blood. Simple phobias may be referred to as “specific” phobias and, in the population at large. Exposure to the phobic stimulus will almost invariably lead to an immediate anxiety response.

Social phobia is characterized by the persistent fear of social or performance situations in which embarrassment may occur. Typical situations feared or avoided by individuals with social phobia include parties, meetings, eating in front of others, writing in front of others, public speaking, conversations, meeting new people, and other related situations. Exposure to social or performance situations almost invariably provokes an immediate anxiety response, as well as sweating, trembling, racing or pounding heart beat, mental confusion, and a desire to flee. Social avoidance and isolation can also become extreme, especially in the more generalized condition. Alcohol abuse is more commonly associated with social phobia than any other anxiety disorder, and frequently represents an attempt at self medication of social fears.

The methods of the invention are useful for treating a subject in need thereof. A subject in need thereof is a subject having or at risk of having an emotional disorder. In its broadest sense, the terms “treatment” or “to treat” refer to both therapeutic and prophylactic treatments. If the subject in need of treatment is experiencing a condition (i.e., has or is having a particular condition), then “treating the condition” refers to ameliorating, reducing or eliminating one or more symptoms associated with the disorder or the severity of the disease or preventing any further progression of the disease. If the subject in need of treatment is one who is at risk of having a condition, then treating the subject refers to reducing the risk of the subject having the condition.

From an evolutionary point of view, mechanisms that impair extinction seem reasonable since rapid extinction of fear after exposure to threatening situations, such as places where a predator attacked, could be disadvantageous for survival. In fact, in contrast to other experimental paradigms where extinction sometimes occurs in a single trial⁴⁸, in the fear conditioning paradigm employed in the examples herein, mice only extinct the aversive freezing behavior after 4-6 extinction trials. This is in line with the fact that patients suffering from emotional disorders such as phobia usually require multiple exposure sessions before the aversively-motivated behavior is extinguished⁴. As such, molecular mechanisms that impair extinction, such as Rac-1 and Cdk5 activity, should be useful targets for emotional disorders.

A subject shall mean a human or vertebrate animal or mammal including but not limited to a dog, cat, horse, cow, pig, sheep, goat, turkey, chicken, and primate, e.g., monkey. Subjects are those which are not otherwise in need of an agent for targeting the Rac1, Cdk5, p35, PAK-1 pathway.

The therapeutic compounds of the invention may be directly administered to the subject or may be administered in conjunction with a delivery device or vehicle. Delivery vehicles or delivery devices for delivering therapeutic compounds to surfaces have been described. The therapeutic compounds of the invention may be administered alone (e.g., in saline or buffer) or using any delivery vehicles known in the art. For instance, the following delivery vehicles have been described: Cochleates; Emulsomes, ISCOMs; Liposomes; Live bacterial vectors (e.g., Salmonella, Escherichia coli, Bacillus calmatte-guerin, Shigella, Lactobacillus); Live viral vectors (e.g., Vaccinia, adenovirus, Herpes Simplex); Microspheres; Nucleic acid vaccines; Polymers; Polymer rings; Proteosomes; Sodium Fluoride; Transgenic plants; Virosomes; Virus-like particles. Other delivery vehicles are known in the art and some additional examples are provided below.

The term effective amount of a therapeutic compound of the invention refers to the amount necessary or sufficient to realize a desired biologic effect. For example, as discussed above, an effective amount of a therapeutic compounds of the invention is that amount sufficient to re-establish access to a memory. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular therapeutic compounds being administered the size of the subject, or the severity of the disease or condition. One of ordinary skill in the art can empirically determine the effective amount of a particular therapeutic compounds of the invention without necessitating undue experimentation.

Subject doses of the compounds described herein for delivery typically range from about 0.1 μg to 10 mg per administration, which depending on the application could be given daily, weekly, or monthly and any other amount of time therebetween. The doses for these purposes may range from about 10 μg to 5 mg per administration, and most typically from about 100 μg to 1 mg, with 2-4 administrations being spaced days or weeks apart. In some embodiments, however, parenteral doses for these purposes may be used in a range of 5 to 10,000 times higher than the typical doses described above.

The formulations of the invention are administered in pharmaceutically acceptable solutions, which may routinely contain pharmaceutically acceptable concentrations of salt, buffering agents, preservatives, compatible carriers, and optionally other therapeutic ingredients.

For use in therapy, an effective amount of the therapeutic compounds of the invention can be administered to a subject by any mode that delivers the therapeutic agent or compound to the desired surface, e.g., mucosal, systemic. Administering the pharmaceutical composition of the present invention may be accomplished by any means known to the skilled artisan. Preferred routes of administration include but are not limited to oral, parenteral, intramuscular, intranasal, sublingual, intratracheal, inhalation, ocular, vaginal, rectal and intracerebroventricular.

For oral administration, the therapeutic compounds of the invention can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, i.e., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline (Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189). Other polymers that could be used are poly-1,3-dioxolane and poly-1,3,6-tioxocane. Preferred for pharmaceutical usage, as indicated above, are polyethylene glycol moieties.

The location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the therapeutic agent or by release of the biologically active material beyond the stomach environment, such as in the intestine.

To ensure full gastric resistance a coating impermeable to at least pH 5.0 is important. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e., powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the therapeutic agent may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the therapeutic agent either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery of the therapeutic compounds of the invention. The therapeutic agent is delivered to the lungs of a mammal while inhaling and traverses across the lung epithelial lining to the blood stream. Other reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5):143-146 (endothelin-1); Hubbard et al., 1989, Annals of Internal Medicine, Vol. III, pp. 206-212 (a1-antitrypsin); Smith et al., 1989, J. Clin. Invest. 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J. Immunol. 140:3482-3488 (interferon-g and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of therapeutic agent. Typically, each formulation is specific to the type of device employed and may involve the use of an appropriate propellant material, in addition to the usual diluents, and/or carriers useful in therapy. Also, the use of liposomes, microcapsules or microspheres, inclusion complexes, or other types of carriers is contemplated. Chemically modified therapeutic agent may also be prepared in different formulations depending on the type of chemical modification or the type of device employed.

Formulations suitable for use with a nebulizer, either jet or ultrasonic, will typically comprise therapeutic agent dissolved in water at a concentration of about 0.1 to 25 mg of biologically active compound per mL of solution. The formulation may also include a buffer and a simple sugar (e.g., for stabilization and regulation of osmotic pressure). The nebulizer formulation may also contain a surfactant, to reduce or prevent surface induced aggregation of the compound caused by atomization of the solution in forming the aerosol.

Formulations for use with a metered-dose inhaler device will generally comprise a finely divided powder containing the therapeutic agent suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing therapeutic agent and may also include a bulking agent, such as lactose, sorbitol, sucrose, or mannitol in amounts which facilitate dispersal of the powder from the device, e.g., 50 to 90% by weight of the formulation. The therapeutic agent should most advantageously be prepared in particulate form with an average particle size of less than 10 mm (or microns), most preferably 0.5 to 5 mm, for most effective delivery to the distal lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The therapeutic compounds of the invention and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the invention contain an effective amount of a therapeutic compound of the invention optionally included in a pharmaceutically-acceptable carrier. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agents may be delivered to the brain using a formulation capable of delivering a therapeutic agent across the blood brain barrier. One obstacle to delivering therapeutics to the brain is the physiology and structure of the brain. The blood-brain barrier is made up of specialized capillaries lined with a single layer of endothelial cells. The region between cells are sealed with a tight junction, so the only access to the brain from the blood is through the endothelial cells. The barrier allows only certain substances, such as lipophilic molecules through and keeps other harmful compounds and pathogens out. Thus, lipophilic carriers are useful for delivering non-lipohilic compounds to the brain. For instance, DHA, a fatty acid naturally occurring in the human brain has been found to be useful for delivering drugs covalently attached thereto to the brain (Such as those described in U.S. Pat. No. 6,407,137). U.S. Pat. No. 5,525,727 describes a dihydropyridine pyridinium salt carrier redox system for the specific and sustained delivery of drug species to the brain. U.S. Pat. No. 5,618,803 describes targeted drug delivery with phosphonate derivatives. U.S. Pat. No. 7,119,074 describes amphiphilic prodrugs of a therapeutic compound conjugated to an PEG-oligomer/polymer for delivering the compound across the blood brain barrier. Others are know to those of skill in the art.

The therapeutic agents of the invention may be delivered with other therapeutics for treating emotional disorders. For instance, one or more of the agents for targeting the Rac1, Cdk5, p35, PAK-1 pathway may be used in combination with one another.

### EXAMPLES

### Experimental Methods

Animals: Mice (Balb/c, 12 weeks old) were housed under standard conditions with access to food and water ad libitum. All mice were purchased from Taconic (Germantown, N.Y.). Behavior testing was performed in a way that the mice were either housed in a separated compartment but within the testing room or different rooms were used for housing and testing. In the latter case the mice were brought to the testing room before the procedure. Although it appears that the dynamics of extinction behavior are somewhat variable depending on the employed behavior facility, the trends remain the same in different experiments. All experiments were approved by the corresponding Animal Use and Care Committee.

Extinction of contextual freezing behavior: Extinction and reminder shock procedures were performed as described before⁶. In brief, mice were exposed to contextual fear conditioning (3 min context followed by at mild electric foot shock, 2 s, 0.7 mA). In some experiments a tone (30 s, 10 kHz, 75 dB SPL) was presented for 30 s before the shock. Extinction was performed on consecutive days. An extinction trial consisted of a 3 min re-exposure to the conditioning context. In our paradigm significant reduction of freezing was observed during E4. However, we found that this time course depends on the experimental setting.

Surgery/injection: Microcannula were purchased custom made from PlasticOne. The gauge of the guide and injection cannulae was 26 and 28, respectively. To insert cannulae into the dorsal hippocampus the coordinates AP −1.5 mm, lateral 1 mm, depth 2 mm were used as described before¹⁸. To insert cannulae icy the coordinates AP +0.5 mm, lateral 1 mm, depth 2 mm were used. Butyrolactone I and roscovitine were dissolved and injected as described before¹⁸. Rac-1 inhibitor was dissolved in aCSF and delivered bilaterally (0.5 μl/side) over a 1 min period. The correct localization of the cannulae was verified at the end of an experiment by methyleneblue injection. The cis-tronic Herpes Virus expressing GFP and dnPAK-1 or GFP alone was injected into the hippocampus bilaterally (0.5 μl/side) over a 2 min period. DnPAK-1 consisted of the DNA encoding amino acid 83-149 of PAK-1. It has been previously demonstrated that this fragment acts as a dominant negative protein towards PAK-1 and impairs PAK-1 activity in vitro and in vivo²⁸.

Immunoblot analysis: Immunoblot analysis was performed as described before¹⁹. The antibody detecting pPAK-1_{Thr212} was used in a 1:250 dilution.

Anti-PAK-1 was from Santa Cruz (Santa Cruz, Calif.). All other antibodies were used in a 1:1000 dilution. pPAK-1_{Thr423} (Cell Signaling, Danvers, Mass.) synaptophysin (Sigma, St. Louis, Mo.), GAPDH and Rac-1 were from Upstate (Lake Placid, N.Y.), Cdk5 (J3) and p35 (C-19) were from Santa Cruz.

Immunohistochemistry: Immunohistochemical analysis was performed as described before¹⁹. Antibodies were used in a 1:1000 concentration:GFP (Molecular Probes; Eugene, Oreg.), NeuN was from Chemicon (Rosemont, Ill.).

Statistical analysis: The data were analyzed by unpaired Student's T-test. One-way ANOVA (ANalyis Of VAriance) followed by post-hoc Scheffe's test was employed to compare means from several groups at the same time. Data are presented as S.E.M. A detailed description of the statistics employed in this study can be found in DC Howell's Statistical Method for Psychology.

### Example 1

### Inhibition of Cdk5 Activity Facilitates Extinction

To test for a role of Cdk5 activity during extinction of learned fear, mice were implanted with microcannulae into the dorsal hippocampus (i.h.) and trained in the contextual fear conditioning paradigm. Fear conditioning consisted of a 3 min context exposure followed by a single electric foot shock (0.7 ms, constant current, 2 s). Subsequently, all mice were subjected to a daily extinction trial (E) on 6 consecutive days (E1-E6). Each extinction trial consisted of a 3 min re-exposure to the conditioned context without presenting the foot-shock again. It was previously shown that in this extinction paradigm mice usually display a significant reduction of aversive freezing behavior upon 4-6 extinction trials that are performed on consecutive days⁶.

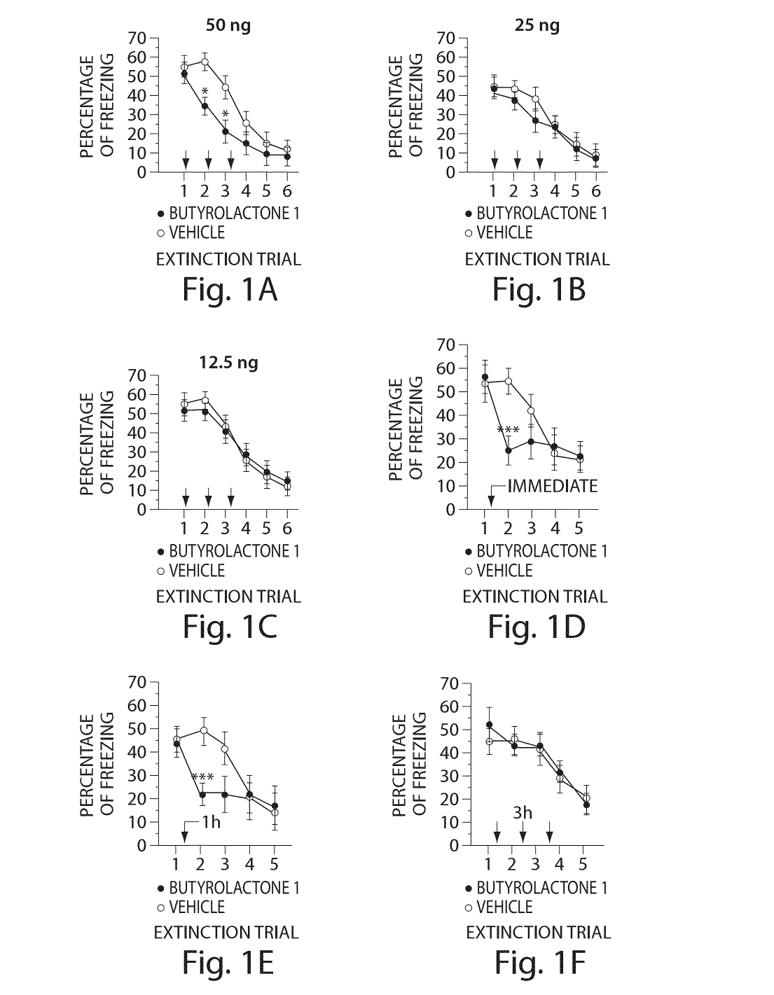

Immediately after the exposure to E1-E3, mice were injected (i.h.) with either vehicle or 3 different concentrations of the Cdk5 inhibitor butyrolactone I. Injection of 50 ng butyrolactone 1 significantly reduced freezing behavior on E2 and E3 when compared to the vehicle group (FIG. 1a). A reminder shock procedure⁶ was able to reinstate freezing behavior implying that the original fear memory was not entirely erased. In agreement with previous data the recovery of the fear response is rarely complete and may indicate that unlearning and new learning may coexist during extinction^{29 30}. The injection of lower concentrations of butyrolactone 1 (25 or 12.5 ng), which did not reduce hippocampal Cdk5 activity as measured by histone H1 phosphorylation and had no significant effect on the reduction of freezing behavior (FIG. 1b, c). Notably, injection of roscovitine (i.h.; 50 ng), another well-established Cdk5 inhibitor, into the dorsal hippocampus immediately after E1-E3 also reduced freezing behavior when compared to a vehicle group. Consistent with the data presented in FIG. 1a, a single injection of butyrolactone I (50 ng) immediately after E1 significantly diminished freezing behavior on the subsequent extinction trials when compared to the vehicle group (FIG. 1d). Similar results were obtained when mice were injected 1 h after E1 (FIG. 1e). In contrast, when butyrolactone I (50 ng) was administered 3 h after E1, freezing behavior was indistinguishable between butyrolactone I- and vehicle-injected mice (FIG. 1f). We estimated that the employed concentrations of butryrolactone I and roscovitine permit a specific inhibition of Cdk5. However high levels of those drugs may also affect Erk-1/2 kinases³¹. Importantly, injection of butyrolactone I (50 ng) into hippocampus did not affect the MAP kinase signaling pathway which is important for extinction of contextual fear^{32,9}. Furthermore, intrahippocampal injection of butyrolactone I did not affect extinction of amygdala-dependent cued fear, demonstrating that Cdk5 inhibition in the hippocampus has a specific effect on contextual fear. Moreover, freezing behavior is not generally affected. In addition, we found that intrahippocampal injection of butyrolactone I 15 min before extinction trial 1 or 2 did not affect freezing behavior during the extinction trial implying that Cdk5 activity affects the consolidation of extinction in our paradigm.

In summary, these data show that inhibition of hippocampal Cdk5 facilitates extinction of aversive freezing behavior. Furthermore, our findings imply that Cdk5 activity is critically involved in regulating extinction within an interval of 1 h after exposure to the extinction trial.

### Example 2

### Increased Cdk5 Activity Impairs Extinction

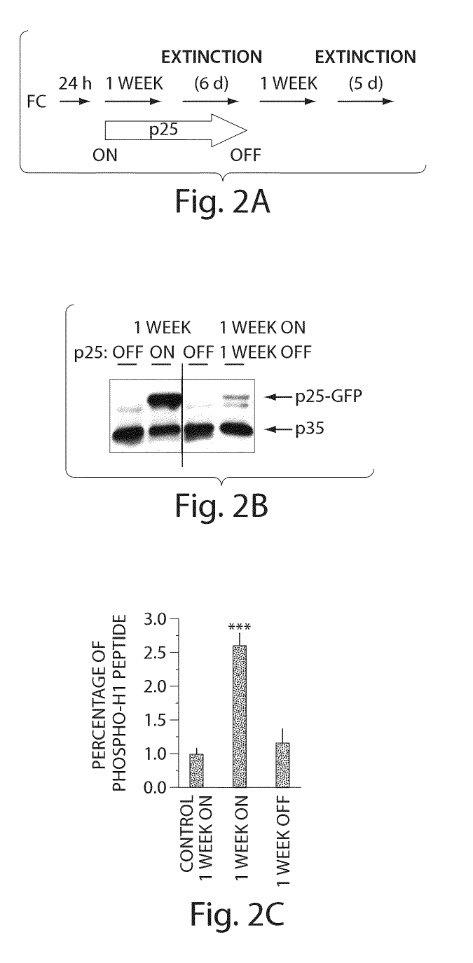

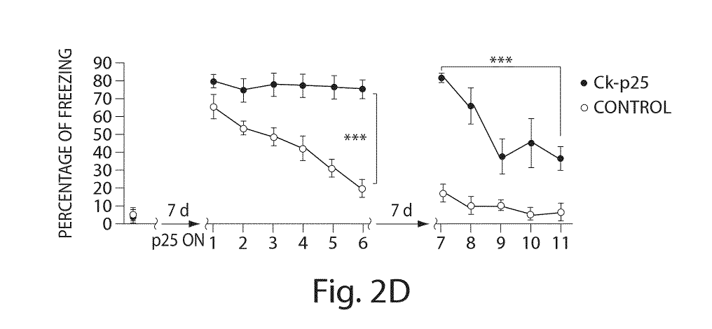

The effect of increased Cdk5 activity on extinction was investigated. To this end CK-p25 Tg mice where forebrain specific expression of the potent Cdk5 activator p25 can be switched on and off by a doxycyline diet¹⁹ were used. We previously showed that expression of p25 for 1-2 weeks increased Cdk5 activity in the hippocampus without causing any neuronal loss¹⁹. CK-p25 Tg and control mice were subjected to contextual fear conditioning. p25 expression was induced 24 h later for 1 week (FIG. 2a, b), which led to increased hippocampal Cdk5 activity (FIG. 2c). At this time point mice were subjected to 6 consecutive extinction trials (see FIG. 2a). In contrast to control mice, no reduction of freezing behavior was observed in CK-p25 Tg mice (FIG. 2d). Next, doxycycline was re-introduced to the diet leading to the repression of p25 expression (FIG. 2b). Consistently hippocampal Cdk5 activity declined to baseline levels when measured 1 week later (FIG. 2c; see 1 week On 1 week Off group). Subsequently all mice were exposed to 5 additional extinction trials. Remarkably, the 1 wkON/1 wkOFF CK-p25 Tg showed a significant reduction in freezing behavior (FIG. 2d). Thus, increased Cdk5 activity prevents extinction, which can be reversed upon down-regulation of Cdk5 activity to baseline levels, and Cdk5 activity negatively regulates extinction.

### Example 3

### Extinction Depletes Cdk5 Activity from the Membranes

To gain further insight into the mechanisms by which Cdk5 regulates extinction, we measured Cdk5/p35 levels during extinction. To this end E1 vs. E3 were compared because in our paradigm the molecular mechanisms activated after E1 (mice show no extinction during subsequent E2) vs. E3 (mice show significant extinction during subsequent E4) might be different. Based on our previous observation that Cdk5 activity is required for extinction within 1 h after an extinction trial (see FIG. 1d-f) we focused the molecular analysis on the time-point 0.5 h after an extinction trial.

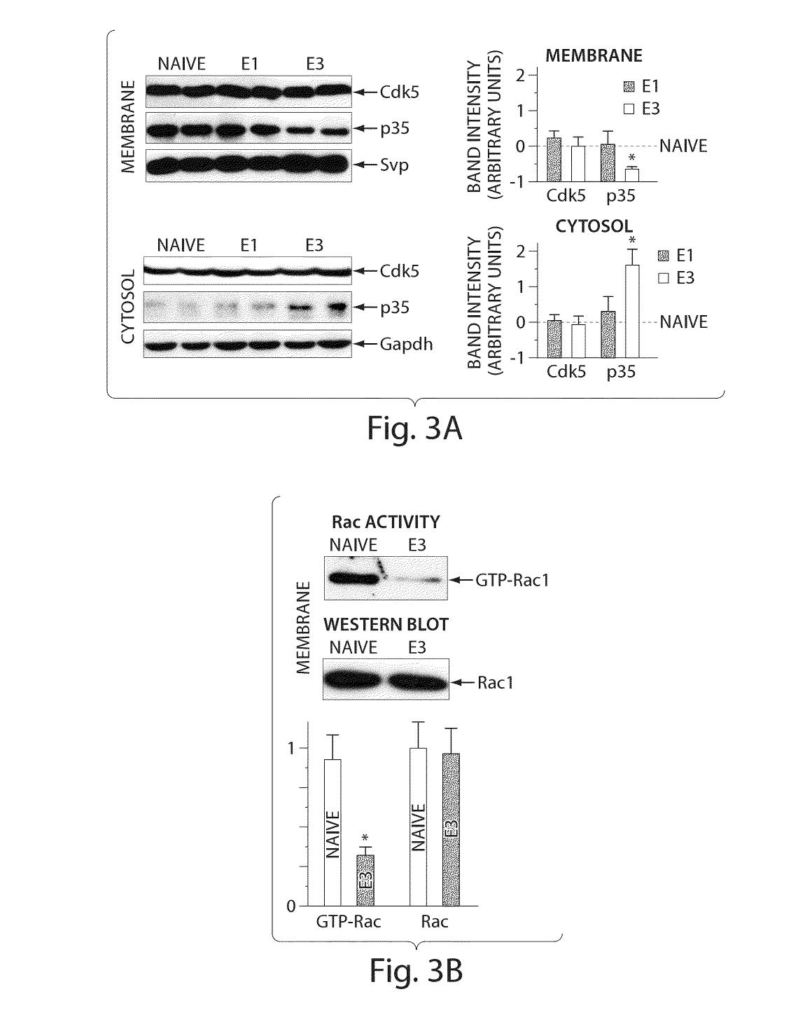

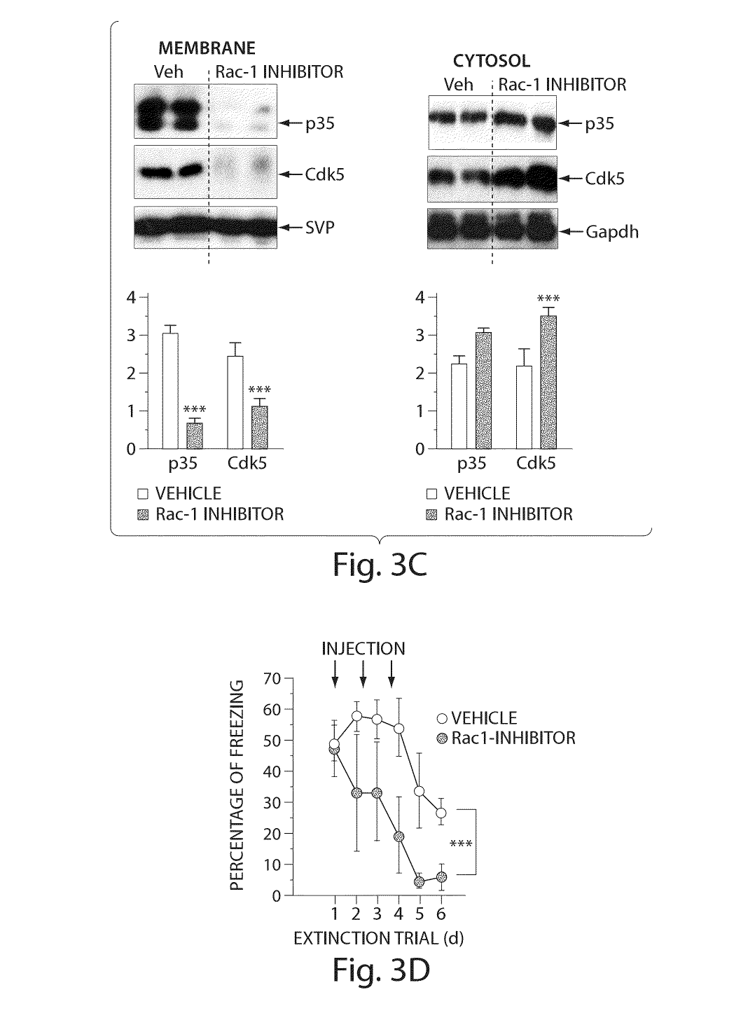

Immunoblot analysis of total protein-lysates prepared from the hippocampus 0.5 h after E1, E3 or from naïve mice revealed no significant difference in p35/Cdk5 levels. We next examined the subcelluar distribution of Cdk5/p35 during extinction. Hippocampal lysates were prepared 0.5 h after E1, E3 and from naive animals and separated into membrane and non-membrane fractions. Notably, we found that membrane associated p35 levels were significantly reduced after E3 when compared to E1 (FIG. 3a).

Importantly, when mice were exposed to E1 and hippocampal protein lysates were prepared 2 days later without any further extinction trial, Cdk5 and p35 protein levels and distribution were not affected, implying that membrane-depletion of p35 is specific to extinction. Taken together, these data show that the reduced freezing behavior during extinction correlates with a reduction of membrane associated p35 levels.

### Example 4

### Rac-1 Regulates the Localization of Cdk5/p35 and Extinction

The small GTPase Rac-1 was shown to regulate the localization and activity of Cdk5/p35 during neurite outgrowth³³. This prompted us to investigate the involvement of Rac-1 in Cdk5 mediated extinction. Rac-1 is only active in its GTP bound form (GTP-Rac-1)³⁴. By employing a Rac-1 activity assay (Upstate) we found that Rac-1 activity was markedly reduced in membrane fraction after E3 when compared to the naïve group (FIG. 3b). Total protein levels of Rac-1 were not significantly different between naïve and E3 implying that membrane associated Rac-1 activity decreased after E1. The correlation between reduction of membrane-associated Rac-1 activity and p35 levels after E3 prompted us to test the possibility of a direct relationship of Rac-1 activity and p35 localization during extinction.

To this end, microcannuale were implanted into the lateral ventricles of mice that were subsequently trained by contextual fear conditioning and exposed to our extinction paradigm. Immediately after E1-E3 mice were injected with a Rac-1 inhibitor³⁵ (NSC23760; 10 μg/μl) or vehicle. Hippocampal membrane and cytosolic fractions were prepared 0.5 h after E3 and analyzed by immunoblot. When compared to the lysates prepared from the vehicle-injected group, mice that were administered with the Rac-1 inhibitor showed a dramatic redistribution of membrane associated p35 and Cdk5 to the cytosol (FIG. 3c). This result implies that Rac-1 activity regulates the distribution of p35/Cdk5 during extinction.

Next we wanted to determine the effect of Rac-1 on extinction directly. Microcannuale were implanted into the lateral ventricles of mice that were subsequently trained by contextual fear conditioning and exposed to our extinction paradigm. Immediately after E1-E3 mice were injected with a Rac-1 inhibitor (NSC23760; 10 μg/μl) or vehicle. Notably, mice injected with the Rac-1 inhibitor displayed facilitated extinction as indicated by a significant reduction of freezing behavior when compared to the vehicle group (FIG. 3d). Similar results were obtained by intrahippocampal injections. Thus, although it is difficult to directly compare the effects of butyrolactone I and Rac-I inhibitors on the dynamics of extinction in absolute terms, similar to Cdk5, Rac-1 activity inhibits extinction.

### Example 5

### A Rac-1-Cdk5-PAK-1 Pathway Regulates Extinction

Cdk5/p35 was previously shown to regulate p21-activated kinase (PAK-1) in a Rac-1 dependent manner^{36 33}. Since PAK-1 is implicated in the dynamics of actin cytoskeleton, learning and synaptic remodeling in the adult brain²⁸, we sought to examine whether PAK-1 plays a role in Cdk5-mediated extinction.

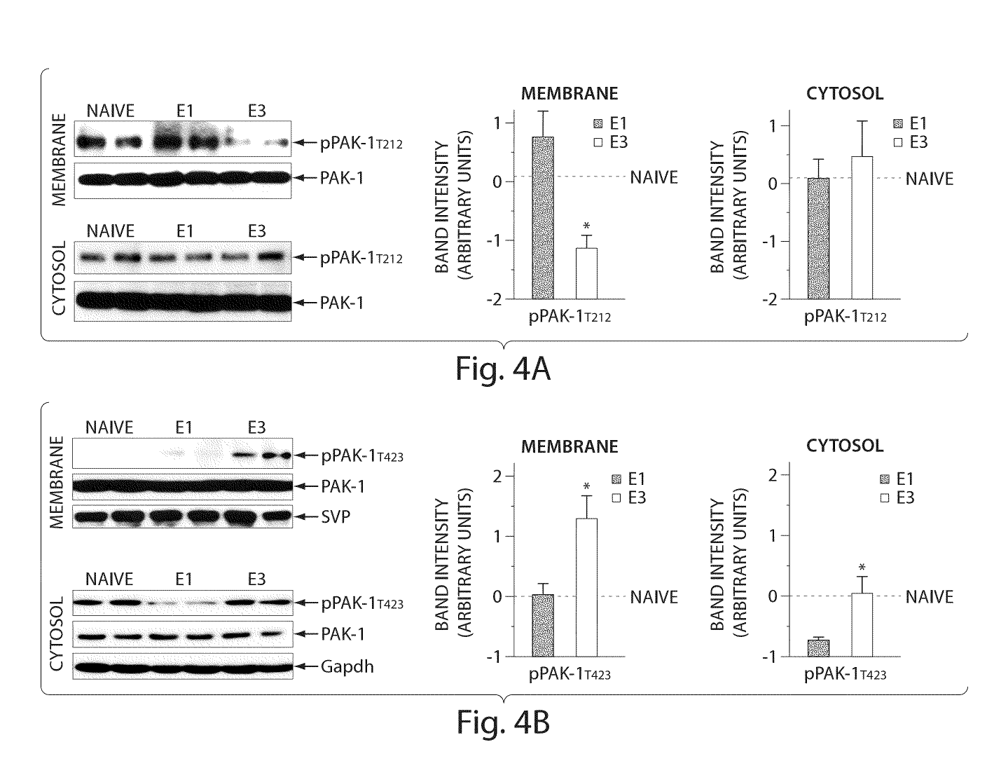

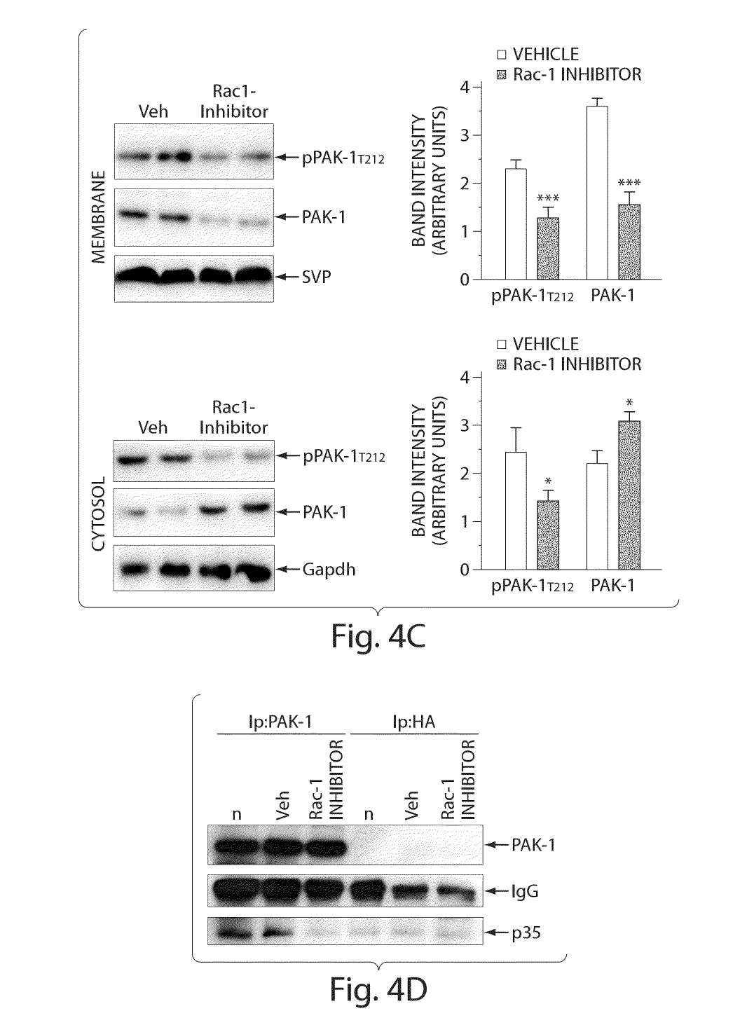

First, we measured PAK-1 phosphorylation at threonine 212 during extinction because this site is phosphorylated by p35/Cdk5³³. We found that PAK-1_{T212} levels were markedly reduced in the E3-membrane fraction when compared to naïve mice or E1 (FIG. 4a). These data are in line with decreased membrane associated p35 levels observed after E3 (see FIG. 3a) and further support the view that Cdk5 activity declines during extinction. We also analyzed the activity of PAK-1 using a phospho-T423 antibody against PAK-1³⁷ in hippocampal lysates prepared from naïve mice and from mice 0.5 h after E1 or E3 by immunoblotting. We detected a significant increase in membrane associated PAK-1_{Thr423} levels after E3, when compared to E1 (FIG. 4b). In the cytosolic fraction PAK-1_{Thr423} levels were down-regulated after E1 when compared to naïve mice and increased after E3 when compared to E1 (FIG. 4b). This data imply that the activity of PAK-1 is up-regulated during extinction.

Next, we set out experiments to determine if the regulation of PAK-1 activity during extinction depended on the activities of Rac-1 and Cdk5/p35. To this end, lysates prepared from mice that were injected with a Rac-1 inhibitor or vehicle (as described under FIG. 3c) were analyzed by immunoblot. When compared to the vehicle-injected group, mice that were administered with the Rac-1 inhibitor showed a dramatic reduction of membrane associated PAK-1 and PAK-1_{T212} levels. Consistently, PAK-1 levels increased in the cytosol of mice treated with Rac-1 inhibitor. However, no corresponding increase in p-PAK-1_{T212} levels was observed in the cytosol. In fact, cytosolic PAK-1_{T212} levels were also decreased. Upon co-immunoprecipitation of PAK-1 and p35 from cytosolic fractions, we found that Rac-1 inhibitor treatment resulted in reduced amount of p35 co-immunoprecipitated with PAK-1 when compared to the vehicle group. This result implies that cytosolic Cdk5/p35 is sequestered from PAK-1 upon Rac-1 inhibition during extinction. In summary these data imply that Rac-1 activity regulates PAK-1 during extinction.

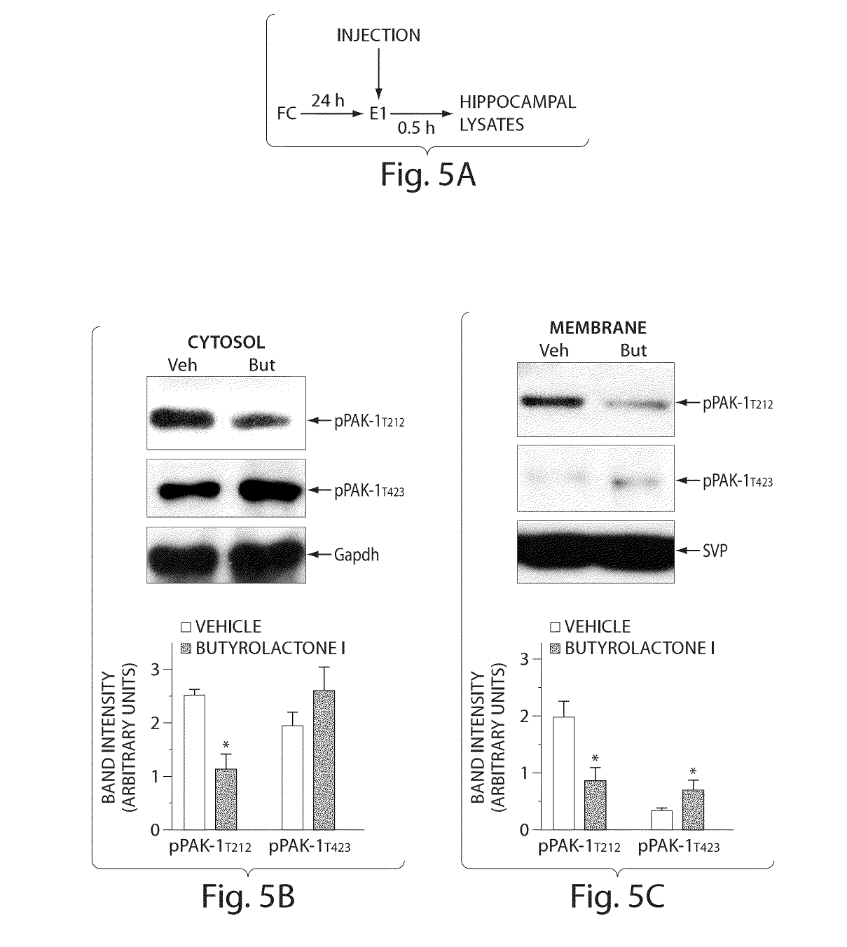

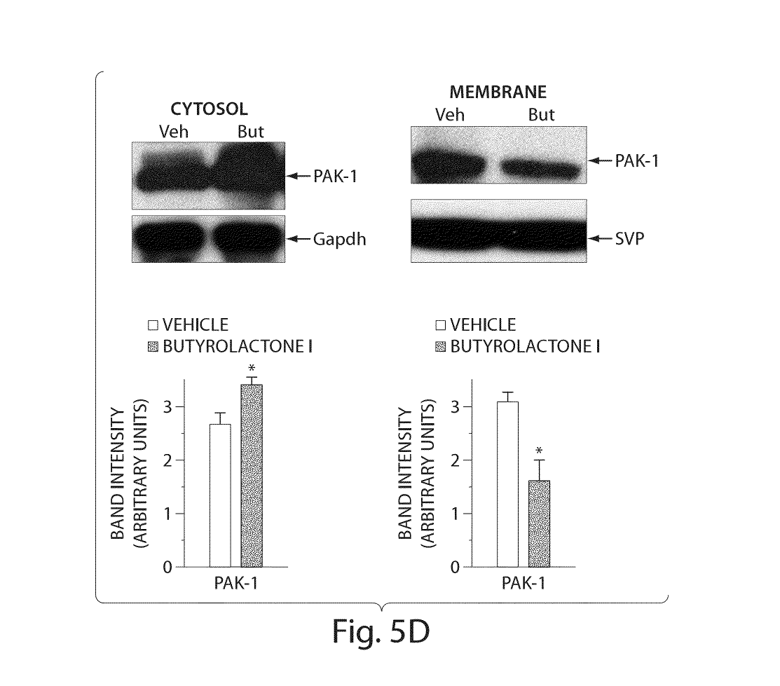

To investigate the impact of Cdk5/p35 activity on PAK-1 function during extinction, mice were implanted with microcannulae into the lateral ventricles and injected with 50 ng butyrolactone I immediately after E1. Because a single injection of butyrolactone I after E1 was sufficient to facilitate extinction (see FIG. 1d), hippocampal lysates were prepared 0.5 h after E1 and analyzed by immunoblotting using PAK-1 antibodies (FIG. 5a). Butyrolactone I injection significantly reduced Cdk5-dependent phosphorylation of PAK-1, since PAK-1_{T212} levels in both the membrane and cytosolic fractions were decreased (FIG. 5b, c). In contrast, PAK-1_{T423} levels increased in lysates obtained from butyrolactone I injected mice (FIG. 5 b, c). Moreover, similar to treatment with Rac-1 inhibitor, inhibition of Cdk5 caused a re-distribution of total PAK-1 from the membrane to the cytosol (FIG. 5d). These data provide evidence that Rac-1 and p35/Cdk5 regulate the activity of PAK-1 during extinction.

### Example 6

### PAK-1 Promotes Extinction

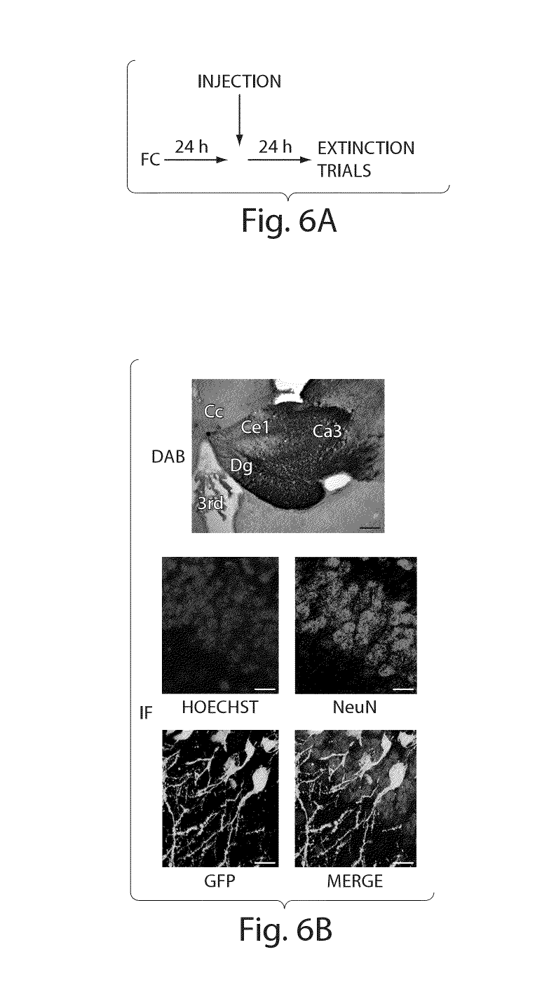

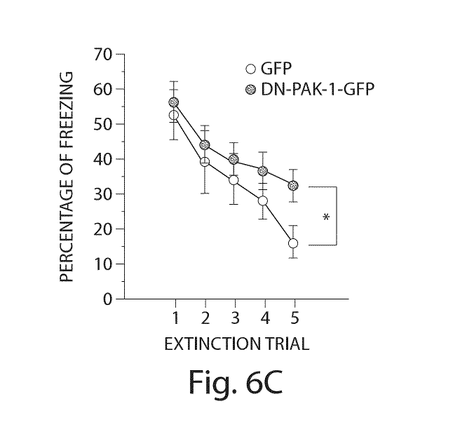

To further determine a role for PAK-1 activity during extinction we introduced a herpes virus expressing GFP and a dominant negative PAK-1 (HSV-dnPAK-1) into the dorsal hippocampus via a microcannulae. It was previously shown that the dnPAK-1 protein significantly inhibits Pak-1 activity in vivo²⁸. In order to investigate extinction it is important that mice first normally acquire fear memories and that any molecular manipulation is only applied after the extinction trial. To this end mice were first subjected to contextual fear conditioning. After 24 h HSV-dnPAK-1 or HSV-GFP (control group) was injected into the hippocampus. Because in pilot experiments we found that expression of this construct (as measured by GFP signal) was first detectable 1 day after injection (FIG. 6b), all mice were subjected to extinction trials the next day (FIG. 6a). Notably, we found that mice injected with HSV-dnPAK-1 showed a significant impairment in extinction when compared to the control group (FIG. 6c). These data show that inhibition of PAK-1 activity impairs extinction.

### REFERENCES

1. Myers, K. M. & Davis, M. Behavioral and neural analysis of extinction. Neuron 52, 998-1007 (2002).
2. Davis, M., Ressler, K., Rothbaum, B. O. & Richardson, R. Effects of D-cycloserine on extinction: translation from preclinical to clinical work. Biol Psychiatry 60 (2006).
3. Lattal, K. M., Radulovic, J. & Lukowiak, K. 9YExtinction: does it or doesn't it? The requirement of altered gene activity and new protein synthesis. Biol Psychiatry 60, 344-351 (2006).
4. Sotres-Bayon, F., Cain, C. K. & LeDoux, J. E. Brain mechanisms of fear extinction: historical perspectives on the contribution of prefrontal cortex. Biol Psychiatry 60, 329-336 (2006).
5. Kim, J. J. & Fanselow, M. S. Modality-specific retrograde amnesia of fear. Science 256, 675-7 (1992).
6. Fischer, A., Sananbenesi, F., Schrick, C., Spiess, J. & Radulovic, J. Distinct roles of hippocampal de novo protein synthesis and actin rearrangement in extinction of contextual fear. J Neurosci 24, 1962-1966 (2004).
7. Falls, W. A., Miserendino, M. J. & Davis, M. Extinction of fear-potentiated startle: blockade by infusion of an NMDA antagonist into the amygdala. J Neurosci 12, 854-863 (1992).
8. Chhatwal, J. P., Stanek-Rattiner, L., Davis, M. & Ressler, K. J. Amygdala BDNF signaling is required for consolidation but not encoding of extinction. Nat Neurosci 9, 870-872 (2006).
9. Szapiro, G., Vianna, M. R., McGaugh, J. L., Medina, J. H. & I., I. The role of NMDA glutamate receptors, PKA, MAPK, and CAMKII in the hippocampus in extinction of conditioned fear. Hippocampus, 1 (2003).
10. Marsicano, G. et al. The endogenous cannabinoid system controls extinction of aversive memories. Nature 418, 530-534 (2002).
11. Fukazawa, Y. et al. Hippocampal LTP is accompanied by enhanced F-actin content within the dendritic spine that is essential for late LTP maintenance in vivo. Neuron 38, 447-460 (2003).
12. Matus, A. Actin-based plasticity in dendritic spines. Science 290, 754-758 (2000).
13. Fischer, A., Sananbenesi, F., Spiess, J. & Radulovic, J. Cdk5 in the adult non-demented brain. Curr Drug Targets CNS Neurol Disord 6, 375-381 (2003).
14. Dhavan, R. & Tsai, L. H. A decade of CDK5. Nat Rev Mol Cell Biol 2, 749-59. (2001).
15. Nikolic, M. The role of Rho GTPases and associated kinases in regulating neurite outgrowth. Int J Biochem Cell Biol. 34 (2002).
16. Cheung, Z. H., Fu, A. K. & Ip, N.Y. Synaptic roles of Cdk5: implications in higher cognitive functions and neurodegenerative diseases. Neuron 50, 13-18 (2006).
17. Ohshima, T. et al. Targeted disruption of the cyclin-dependent kinase 5 gene results in abnormal corticogenesis, neuronal pathology and perinatal death. Proc Nall Acad Sci USA 93, 11173-8 (1996).
18. Fischer, A., Sananbenesi, F., Schrick, C., Spiess, J. & Radulovic, J. Cyclin-dependent kinase 5 is required for associative learning. J Neurosci 22, 3700-7. (2002).
19. Fischer, A., Sananbenesi, F., Pang, P. T., Lu, B. & Tsai, L. H. Opposing roles of transient and prolonged expression of p25 in synaptic plasticity and hippocampus-dependent memory. Neuron 48, 825-838 (2005).
20. Angelo, M., Plattner, F., Irvine, E. E. & Giese, K. P. Improved reversal learning and altered fear conditioning in transgenic mice with regionally restricted p25 expression. Eur J Neurosci 18, 423-31 (2003).
21. Li, B. S. et al. Regulation of NMDA receptors by cyclin-dependent kinase-5. Proc Natl Acad Sci USA 98, 12742-7. (2001).
22. Tan, T. C. et al. Cdk5 is essential for synaptic vesicle endocytosis. Nat Cell Biol 5, 701-10 (2003).
23. Tomizawa, K. et al. Cophosphorylation of amphiphysin I and dynamin I by Cdk5 regulates clathrin-mediated endocytosis of synaptic vesicles. J Cell Biol 163, 813-24 (2003).
24. Kim, Y. et al. Phosphorylation of WAVE1 regulates actin polymerization and dendritic spine morphology. Nature 442, 814-817 (2006).
25. Lee, S. Y., Wenk, M. R., Kim, Y., Nairn, A. C., De Camilli, P. Regulation of synpatojanin 1 by cyclin-dependent kinase 5 at synapses. Proc Natl Acad Sci USA 101, 546-51 (2004).
26. Hawasli, A. H. et al. Cyclin-dependent kinase 5 governs learning and synaptic plasticity via control of NMDAR degradation. Nat Neurosci Epub ahead of print (2007).
27. Nakayama, A. Y., Harms, M. B. & Luo, L. Small GTPases Rac and Rho in the maintenance of dendritic spines and branches in hippocampal pyramidal neurons. J. Neurosci 20, 5329-5338 (2000).
28. Hayashi, M. L. et al. Altered cortical synaptic morphology and impaired memory consolidation in forebrain-specific dominant-negative PAK transgenic mice. Neuron 42, 773-87 (2004).
29. Delamater, A. R. Experimental extinction in Pavlovian conditioning: behavioural and neuroscience perspectives. Q J Exp Psychol B. 57, 97-132 (2004).
30. Myers, K. M. & Davis, M. Mechanisms of fear extinction. Mol Psychiatry 12, 120-150 (2007).
31. Kitagawa, M. et al. Butyrolactone I, a selective inhibitor of cdk2 and cdc2 kinase. Oncogene 8, 2425-2432 (1993).
32. Fischer, A. et al. Hippocampal Mek/Erk signaling mediates extinction of contextual freezing behavior. Neurobiol Learn Mem. 87, 149-158 (2007).
33. Nikolic, M., Chou, M. M., Lu, W., Mayer, B. J. & Tsai, L. H. The p35/Cdk5 kinase is a neuron-specific Rac effector that inhibits Pak1 activity. Nature 395, 194-8. (1998).
34. Zhang, Q. G. et al. Akt inhibits MLK3/JNK3 signaling by inactivating Rac1: a protective mechanism against ischemic brain injury. J Neurochem 98, 1886.1898 (2006).
35. Desire, L. et al. RAC1 inhibition targets amyloid precursor protein processing by gamma-secretase and decreases Abeta production in vitro and in vivo. J Biol Chem 280, 37516-37525 (2005).
36. Rashid, T., Banerjee, M. & M., N. Phosphorylation of Pak1 by the p35/Cdk5 kinase affects neuronal morphology. J Biol Chem 276, 49043-49052 (2001).
37. Lei, M. et al. Structure of PAK1 in an autoinhibited conformation reveals a multistage activation switch. Cell 102, 387-397 (2000).
38. Wong, S. T. et al. Calcium-stimulated adenylyl cyclase activity is critical for hippocampus-dependent long-term memory and late phase LTP. Neuron 23, 787-798 (1999).
39. Wang, H., Ferguson, G. D., Pineda, V. V., Cundiff, P. E. & Storm, D. R. Overexpression of type-1 adenylyl cyclase in mouse forebrain enhances recognition memory and LTP. Nat Neurosci 7, 635-642 (2004).
40. Burgos-Robles, A., Vidal-Gonzalez, I., Santini, E. & Quirk, G. J. Consolidation of fear extinction requires NMDA receptor-dependent bursting in the ventromedial prefrontal cortex. Neuron 53, 871-880 (2007).
41. Vianna, M. R., Coitinho, A. S. & Izquierdo, I. Role of the hippocampus and amygdala in the extinction of fear-motivated learning. Curr Neurovasc Res 1, 55-60 (2004).
42. Berlau, D. J. & McGaugh, J. L. Enhancement of extinction memory consolidation: the role of the noradrenergic and GABAergic systems within the basolateral amygdala. Neurobiol Learn Mem 86, 123-132 (2006).
43. Lin, C. H. et al. Identification of calcineurin as a key signal in the extinction of fear memory. J. Neurosci 23, 1574-1579 (2003).
44. Bonhoeffer, T. & Yuste, R. Spine motility. Phenomenology, mechanisms, and function. Neuron 35, 1019-1027 (2002).
45. Edwards, D. C., Sanders, L. C., Bokoch, G. M. & Gill, G. N. Activation of LIM-kinase by Pak1 couples Rac/Cdc42 GTPase signalling to actin cytoskeletal dynamics. Nat Cell Biol 1, 253-259 (1999).
46. Arber, S. et al. Regulation of actin dynamics through phosphorylation of cofilin by LIM-kinase. Nature 393, 739-740 (1998).
47. Fu, W. Y. et al. Cdk5 regulates EphA4-mediated dendritic spine retraction through an ephexinl-dependent mechanism. Nat Neurosci 10, 67-76 (2007).
48. Berman, D. E. & Dudai, Y. Memory extinction, learning anew, and learning the new: dissociations in the molecular machinery of learning in cortex. Science 291, 2417-2419 (2001).
49. Howell, D. C. Statistical Methods for Psychology (ed. Crokett, C.) (Duxbury Thompson Learning, Duxbury, 2002).

This invention is not limited in its application to the details of construction and the arrangement of components set forth in the description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A method for treating an emotional disorder by administering to a subject an effective amount of an agent for targeting the Rac1, Cdk5, p35, PAK-1 pathway to treat the emotional disorder.

2. The method of claim 1 wherein the emotional disorder is a stress induced emotional disorder.

3. A method for treating an emotional disorder comprising
administering to a subject an effective amount of a Rac-1 inhibitor to treat the emotional disorder.

4. The method of claim 3, wherein the Rac-1 inhibitor is a direct or an indirect Rac-1 inhibitor.

5. The method of claim 3, wherein the Rac-1 inhibitor is a Rac-1 activity inhibitor or a Rac-1 expression inhibitor.

6. The method of claim 3, wherein the Rac-1 inhibitor is NSC23760.

7. The method of claim 3, further comprising administering a second therapeutic, selected from the group consisting of a Cdk5 inhibitor and a PAK-1 activator.

8. A method for treating an emotional disorder comprising
administering to a subject an effective amount of a Cdk5 inhibitor to treat the emotional disorder.

9. The method of claim 8, wherein the Cdk5 inhibitor is a direct or an indirect Cdk5 inhibitor.

10. The method of claim 9, wherein the indirect Cdk5 inhibitor is a Rac-1 inhibitor.

11. The method of claim 8, wherein the Cdk5 inhibitor is a Cdk5 activity inhibitor or a CDK5 expression inhibitor.

12. The method of claim 8, wherein the Cdk5 inhibitor is butyrolactone or roscovitine.

13. The method of claim 8, further comprising administering a second therapeutic, selected from the group consisting of a Rac1 inhibitor and a PAK-1 activator.

14. A method for treating an emotional disorder comprising
administering to a subject an effective amount of a PAK-1 activator to treat the emotional disorder.

15. The method of claim 14, wherein the PAK-1 activator is a direct or an indirect PAK-1 activator.

16. The method of claim 15, wherein the indirect PAK-1 activator is a Cdk5 inhibitor or a Rac-1 inhibitor.

17. The method of claim 14, wherein the PAK-1 activator is a PAK-1 activity activator or a PAK-1 expression activator.

18. The method of claim 14, further comprising administering a second therapeutic, selected from the group consisting of a Rac1 inhibitor and a Cdk5 inhibitor.

19. A method for treating an emotional disorder comprising
administering to a subject a p35 mobilizing agent in an effective amount to mobilize the p35 to the cytosol membrane, to treat the emotional disorder.


SEQUENCE LISTING (1 sequence)

SEQ ID NO: 1 - protein, 16 aa, Artificial sequence
  Synthetic peptide
MVDGKPVNLGLWDTAG